(19) United States
(12) Patent Application Publication
(10) Pub. No.: US 2006/0019871 A1
(43) Pub. Date: Jan. 26, 2006

(54) Endothelial cell apoptosis induced by fibrinogen gamma chain C-terminal fragment

(75) Inventors: Yoshikazu Takada, Davis, CA (US); Nobuaki Akakura, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/123,817

(22) Filed: May 6, 2005

Related U.S. Application Data
(60) Provisional application No. 60/569,002, filed on May 7, 2004.

(51) Int. Cl. A61K 38/18

(52) U.S. Cl. 514/2

(57) ABSTRACT

The present invention provides for the novel use of a polypeptide related to a fibrinogen γ chain C-terminal fragment or a nucleic acid encoding the polypeptide for inhibiting endothelial cell proliferation. Methods of using the polypeptide or the nucleic acid are provided. Also provided are compositions containing the polypeptide or the nucleic acid and kits containing the compositions.

### RELATED PATENT APPLICATIONS

This application claims priority to U.S. provisional application No. 60/569,002, filed May 7, 2004, the contents of which are hereby incorporated by reference in the entirety.

### STATEMENT AS TO RIGHTS TO INVENTIONS MADE UNDER FEDERALLY SPONSORED RESEARCH AND DEVELOPMENT

This invention was made with government support under Grant No. GM49899 by the National Institutes of Health. The government has certain rights in this invention.

### BACKGROUND OF THE INVENTION

Angiogenesis, the process of blood vessel formation, is a key event in many physiological processes that underlie normal and diseased tissue function. This process of blood vessel formation relies on the proliferation of endothelial cells, which line the lumen of blood vessels. During ontogeny, angiogenesis is necessary to establish to the network of blood vessels required for normal cell, tissue, and organ development and maintenance. In the adult organism, the production of new blood vessels is needed for organ homeostasis, e.g., in the cycling of the female endometrium, for blood vessel maturation during wound healing, and other processes involved in the maintenance of organism integrity. It also is important in regenerative medicine, including, e.g., in promoting tissue repair, tissue engineering, and the growth of new tissues, inside and outside the body.

Not all angiogenesis is beneficial. Inappropriate and ectopic angiogenesis can be deleterious to an organism. A number of pathological conditions are associated with the growth of extraneous blood vessels. These include, e.g., diabetic retinopathy, neovascular glaucoma, psoriasis, retrolental fibroplasias, angiofibroma, and inflammation. In addition, the increased blood supply associated with cancerous and neoplastic tissue encourages growth, leading to rapid tumor enlargement and metastasis.

Numerous approaches have been taken to regulate angiogenesis. For instance, induction of neoangiogenesis has been used for the treatment of ischemic myocardial diseases, and other conditions (e.g., ischemic limb, stroke) produced by the lack of adequate blood supply. See, e.g., Rosengart et al., Circulation, 100(5):468-74, 1999. Angiogenesis is one of the key processes necessary for supporting the growth of new tissues from progenitor and stem cells. Where vascularization is undesirable, such as for cancer and the pathological conditions mentioned above, inhibition of angiogenesis has been used as a treatment therapy. See, e.g., U.S. Pat. Nos. 5,994,388; 6,024,688; 6,174,861; 6,242,481; 6,380,203; 6,413,513; 6,525,019; 6,548,477; 6,573,096; 6,589,979; and 6,673,843 for compositions and methods for inhibiting angiogenesis.

A number of different factors have been identified that stimulate angiogenesis, e.g., by activating normally quiescent endothelial cells, by acting as a chemo-attractant to developing capillaries or by stimulating gene expression. These factors include, e.g., fibroblast growth factors, such as FGF-1 and FGF-2, vascular endothelial growth factor (VEGF), and platelet-derived endothelial cell growth factor (PD-ECGF).

Inhibition of angiogenesis has been achieved using drugs, such as TNP-470, monoclonal antibodies, antisense nucleic acids, and proteins, such as angiostatin and endostatin. See, e.g., Battegay, J. Mol. Med., 73:333-346 (1995); Hanahan et al., Cell 86:353-364 (1996); Folkman, N. Engl. J. Med. 333:1757-1763 (1995).

Because of the importance of angiogenesis, particularly its involvement in tumor biology, there remains a need to develop new strategies for regulating angiogenesis. The present invention addresses this and other needs.

### BRIEF SUMMARY OF THE INVENTION

This invention provides new methods and compositions useful for suppressing the proliferation of endothelial cells and therefore undesired angiogenesis, based on the surprising discovery that the carboxyl terminal fragment of fibrinogen γ chain (fibrinogen γC), but not the entire γ chain, has an anti-proliferative effect on endothelial cells. Thus, in one aspect, the present invention relates to a composition comprising a fibrinogen γC-related polypeptide and a pharmaceutically acceptable carrier. This polypeptide has two basic properties: first, it contains an amino acid sequence that has at least 90% sequence identity to the full length of SEQ ID NO:3, 4, or 6; and second, it can inhibit endothelial cell proliferation.

In some embodiments, the fibrinogen γC-related polypeptide inhibits endothelial cell proliferation in an in vitro assay. In other embodiments, the amino acid sequence is SEQ ID NO:3, SEQ ID NO:4, or SEQ ID NO:6. In one example, this amino acid sequence is the sequence of 1-249 of SEQ ID NO:3. In some embodiments, the administering is performed locally, such as direct delivery into an organ suffering from a condition exacerbated by the continued proliferation of endothelial cells (e.g., direct injection into a tumor). In other embodiments, the composition is a part of a kit for inhibiting endothelial cell proliferation.

In a second aspect, the present invention relates to a composition comprising a nucleic acid and a pharmaceutically acceptable carrier. This nucleic acid includes a polynucleotide sequence, which encodes a fibrinogen γC-related polypeptide that: (a) comprises an amino acid sequence having at least 90% sequence identity to the full length of SEQ ID NO:3, 4, or 6; and (b) inhibits endothelial cell proliferation.

In some embodiments, the fibrinogen γC-related polypeptide inhibits endothelial cell proliferation in an in vitro assay. In other embodiments, the amino acid sequence is SEQ ID NO:3, SEQ ID NO:4, or SEQ ID NO:6. In one example, this amino acid sequence is a subsequence of SEQ ID NO:3 (amino acid residues 1-249, inclusive). In other embodiments, the administering is performed locally. In yet other embodiments, this claimed composition is a part of a kit for inhibiting endothelial cell proliferation.

In a third aspect, the present invention relates to a method for inhibiting endothelial cell proliferation. This method includes the step of contacting an endothelial cell an effective amount of a fibrinogen γC-related polypeptide. This invention also relates to a method for inhibiting endothelial cell proliferation in a patient, including the step of administering to the patient an effective amount of a fibrinogen γC-related polypeptide. In both methods, the fibrinogen γC-related polypeptide is characterized as: (a) containing an amino acid sequence having at least 90% sequence identity to the full length of SEQ ID NO:3, 4, or 6; and (b) capable of inhibiting endothelial cell proliferation.

In other embodiments, this claimed composition is a part of a kit for inhibiting endothelial cell proliferation. In some embodiments, the fibrinogen γC-related polypeptide inhibits endothelial cell proliferation in an in vitro assay. In other embodiments, the amino acid sequence is SEQ ID NO:3, SEQ ID NO:4, or SEQ ID NO:6. In one example, this amino acid sequence is amino acids 1-249 of SEQ ID NO:3. In yet other embodiments, the administering is performed locally.

In a fourth aspect, the present invention relates to a method for inhibiting endothelial cell proliferation. This method includes the step of contacting an endothelial cell an effective amount of a nucleic acid comprising a polynucleotide, which encodes a fibrinogen γC-related polypeptide. This invention also relates to a method for inhibiting endothelial cell proliferation in a patient, which includes the step of administering to the patient an effective amount of a nucleic acid comprising a polynucleotide, which encodes a fibrinogen γC-related polypeptide. In both methods, the fibrinogen γC-related polypeptide comprises an amino acid sequence having at least 90% sequence identity to the full length of SEQ ID NO:3, 4, or 6 and is capable of inhibiting endothelial cell proliferation.

In some embodiments, the fibrinogen γC-related polypeptide inhibits endothelial cell proliferation in an in vitro assay. In other embodiments, the amino acid sequence is SEQ ID NO:3, SEQ ID NO:4, or SEQ ID NO:6. In one example, this amino acid sequence is amino acids 1-249 of SEQ ID NO:3. In yet other embodiments, the administering is performed locally.

### BRIEF DESCRIPTION OF THE DRAWINGS

FIG. 1. Fibrinogen γC-induced growth arrest of Bovine Arterial Endothelial (BAE) cell. BAE cells were plated in wells of a 96-well tissue culture plate at 1×10⁴ cells per well in the presence of soluble native fibrinogen, fragment D or γC (12.5 μg/ml each) in the culture media. γC blocked proliferation of BAE cells as shown (pictures were taken after 48 hours). Native fibrinogen or fragment D did not induce such effects under the conditions used. Fragment D required much higher concentrations (100 μg/ml) to induce detectable apoptotic effects.

FIG. 2. Fibrinogen γC blocked proliferation of BAE cells, but did not block proliferation of CHO cells. The numbers of proliferating cells were determined using a tetrazolium compound MTS (CellTiter96® assay). The data show that γC effectively block proliferation of BAE cells at very low levels of γC (below 1 μg/ml). In contrast, γC showed little or no effect on the proliferation of CHO or β3-CHO cells. This indicates that γC's anti-proliferative effect is specific to endothelial cells.

FIGS. 3A and 3B. Fibrinogen γC induced apoptosis of BAE cells. BAE cells were treated with 10 μg/ml recombinant soluble γC or native fibrinogen for the indicated time. The binding of annexin V or propidium iodide (PI) to the treated BAE cells was measured in flow cytometry (FIG. 3A). Cells in the upper windows (PI-high) represent dead cells, and cells in the lower right window (PI-low, annexin V binding-high) represent early apoptotic cells. γC-induced apoptosis of BAE cells was detectable in 2-4 h (FIG. 3B). Native fibrinogen did not induce such effects.

FIG. 4. Fibrinogen γC-induced activation of MAP kinases Erk1 and 2. CHO cells expressing recombinant αvβ3 (β3-CHO cells) were incubated with soluble γC at indicated concentrations for 30 min and the levels of MAP kinases (Erk1 and 2) were determined by western blotting with anti-phosphorylated Erk 1 and 2. The level of total MAP kinase in each lane is comparable.

FIG. 5. Inhibition of CPAE Proliferation induced by fibrinogen γC and γC-399tr. CPAE proliferation was measured by using a MTS and Phosphate assay. 10% serum-induced proliferation was inhibited by γC in a concentration-dependent fasion 48 hours passed after 1×10⁵/ml. The γC-399tr is approximately 3 times more efficient in inducing apoptosis of CPAE cells.

FIG. 6. p38 MAPK inhibitor SB-203580 blocks γC-399tr-induced apoptosis in a dose-dependent fashion. 10 μM of inhibitor is the saturating concentration. Inhibitors to other MAPKs do not block γC-399tr-induced apoptosis.

### DEFINITIONS

The term “inhibiting” or “inhibition,” as used herein, refers to any detectable negative effect on endothelial cell proliferation. Such a negative effect may include the slowing or arrest of cell proliferation as well as the induction of cell death. Typically, an inhibition is reflected in a decrease of at least 10%, 20%, 30%, 40%, or 50% in endothelial cell proliferation, or an increase of at least 10%, 20%, 50%, or 100% in endothelial cell apoptosis, when compared to a control.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N.Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a γC-related amino acid sequence of the present invention has at least 80% identity, preferably 85%, 90%, 91%, 92%, 93, 94%, 95%, 96%, 97%, 98%, 99%, or 100% identity, to a reference sequence, e.g., SEQ ID NO:3, 4, or 6), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to fibrinogen γC nucleic acids and proteins, e.g., SEQ ID NO:1 and SEQ ID NO:3, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of pharmaceutical Compounding (1999); and Pickar, Dosage Calculations (1999)).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

As used herein, a “polypeptide related to fibrinogen γ chain C-terminal fragment” or “γC-related polypeptide” refers to a polypeptide containing a core amino acid sequence that generally corresponds to the carboxyl terminal fragment of a fibrinogen γ chain, the full length of which is exemplified by, e.g., SEQ ID NO:2 or 5. This core γC amino acid sequence may contain some variations such as amino acid deletion, addition, or substitution, but should maintain a substantial level sequence homology (e.g., at least 80%, 85%, 90%, 95%, or higher sequence homology) to a fibrinogen γ chain C-terminal fragment (e.g., SEQ ID NO:3, 4, or 6) capable of suppressing endothelial cell proliferation. Some examples of the core γC amino acid sequence include those that have at least 4 amino acids (AGDV) deleted from the C-terminus of SEQ ID NO:3. Such a deletion from SEQ ID NO:3 can be up to 20 amino acid from the C-terminus, more preferably up to 15 or 12 amino acids. One exemplary core sequence is the 1-249 segment of SEQ ID NO:3. Similarly, a core γC amino acid sequence can be generated from SEQ ID NO:4 by deleting at least 4 amino acids (AGDV) and up to 20 amino acids from the C-terminus of SEQ ID NO:4, more preferably up to 12 or 15 amino acids can be deleted.

Besides the core γC amino acid sequence, the γC-related polypeptide of the present invention may further contain additional amino acid sequence, which can be heterologous in origin (e.g., an epitope tag) or homologous in origin (e.g., additional sequence from a fibrinogen γ chain), but should retain the same functionality, i.e., capable of inhibiting endothelial cell proliferation. A γC-related polypeptide as used in this application does not encompass a full-length fibrinogen γ chain.

### DETAILED DESCRIPTION OF THE INVENTION

I. Introduction

Fibrinogen is a 340 kDa major plasma glycoprotein that consists of two identical disulfide-linked subunits, each of which in turn consists of three different polypeptide chains: α, β, and γ. Fibrinogen is known to play an important role in physiological and pathological processes such as blood clotting, cellular and matrix interactions, inflammation, wound healing, and neoplasia. For instance, the binding between fibrinogen and integrin αvβ3, a member of a class of major cell-surface receptors for extracellular matrix proteins (including fibrinogen), has been shown to promote cell growth in wound healing and tumorigenesis. See, e.g., Brooks et al., Science 264:569-571, (1994); Clark et al., Am. J. Pathol. 148:1407-1421 (1996). It has also been reported that fibrin(ogen)-bound fibroblast growth factor-2 (FGF-2) stimulates the proliferation of endothelial cells. Sahni et al., J Biol Chem. 274:14936-41 (1999).

Integrin αvβ3 is a major receptor for fibrinogen and is found on the surface of a variety of cells including endothelial cells. The interaction between fibrinogen and integrin αvβ3 has been implicated in tumor-induced angiogenesis. The carboxyl terminal domain of fibrinogen γ chain (γC), which consists of about 250 amino acids and has a molecular weight of about 30 kDa, has been shown in previous studies to specifically interact with integrin αvβ3. Yokoyama et al., Biochemistry 38:5872-5877 (1999).

Surprisingly, it has been discovered that, unlike intact fibrinogen, fibrinogen γC induces growth arrest and apoptosis in endothelial cells. While not intending to be bound by any particular theory, it is believed that the cellular signals leading to endothelial cell death are transduced via the activation of MAP kinases such as Erk1 and Erk2 by fibrinogen γC.

An exemplary fibrinogen γC polypeptide of the present invention has the amino acid sequence set forth in SEQ ID NO:3, SEQ ID NO:4, or SEQ ID NO:6. Shorter polypeptides with essentially the same inhibitory effect on endothelial cell proliferation can be readily made and identified by one of skill in the art according to the methods described below. Thus, these shorter polypeptides are also within the contemplation of the present invention. An exemplary shorter γC amino acid sequence is amino acids 1-249 of SEQ ID NO:3, which the present inventors have discovered to be surprisingly effective in inducing programmed cells death in endothelial cells. Other shorter sequences include those with a deletion of at least 4 amino acids and up to 20 amino acids from the C-terminus of SEQ ID NO:3. In some cases, these variants of γC amino acid sequence can be generated from SEQ ID NO:3, 4, or 6 by altering the C-terminal sequence while making no changes in the N-terminal sequence.

In uncovering the activity of fibrinogen γC to suppress endothelial cell proliferation, the present invention provides useful compositions and methods for inhibiting undesirable physiological or pathological processes in which endothelial cell growth plays an important part. The present invention thus offers a novel therapeutic strategy for treating hyperplasia (such as various types of cancer), metastasis, and other diseases that are associated with the growth of extraneous blood vessels (e.g., diabetic retinopathy, neovascular glaucoma, psoriasis, retrolental fibroplasias, angiofibroma, inflammation, rheumatoid arthritis, age-related macular degeneration, etc.)

II. Acquisition of Fibrinogen γC-Related Polypeptides

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a fibrinogen γ chain gene, a polynucleotide encoding a fibrinogen γ chain C-terminal segment, and synthetic oligonucleotides can be verified after cloning or subcloning using, e.g., the chain termination method for sequencing double-stranded templates of Wallace et al., Gene 16: 21-26 (1981).

B. Cloning and Subcloning of a Coding Sequence for Fibrinogen γC

A number of polynucleotide sequences encoding fibrinogen γ chains, e.g., GenBank Accession Nos. BC007044, BC021674, and AF118092 have been determined and may be obtained from a commercial supplier.

The rapid progress in the studies of human genome has made possible a cloning approach where a human DNA sequence database can be searched for any gene segment that has a certain percentage of sequence homology to a known nucleotide sequence, such as one encoding a previously identified human fibrinogen γ chain. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a human fibrinogen γ chain can be isolated from a human cDNA or genomic DNA library using standard cloning techniques such as polymerase chain reaction (PCR), where homology-based primers can often be derived from a known nucleic acid sequence encoding a fibrinogen γC polypeptide. Most commonly used techniques for this purpose are described in standard texts, e.g., Sambrook and Russell, supra.

cDNA libraries suitable for obtaining a coding sequence for a human fibrinogen γ chain may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the fibrinogen γ chain from the cDNA library. A general description of appropriate procedures can be found in Sambrook and Russell, supra.

A similar procedure can be followed to obtain a full-length sequence encoding a human fibrinogen γ chain, e.g., any one of the GenBank Accession Nos. mentioned above, from a human genomic library. Human genomic libraries are commercially available or can be constructed according to various art-recognized methods. In general, to construct a genomic library, the DNA is first extracted from an tissue where a fibrinogen γ chain is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a fibrinogen γ chain is obtained.

Upon acquiring a nucleic acid sequence encoding a fibrinogen γ chain, the coding sequence for the carboxyl terminal region, e.g., the C-terminal 261 amino acids, of the γ chain can be obtained by a number of well known techniques such as restriction endonuclease digestion, PCR, and PCR-related methods. The polynucleotide sequence encoding a fibrinogen γ polypeptide (or a fragment of γC that retains the activity of inhibiting endothelial cell proliferation) can then be subcloned into a vector, for instance, an expression vector, so that a recombinant fibrinogen γC polypeptide can be produced from the resulting construct. Further modifications to the fibrinogen γC coding sequence, e.g., nucleotide substitutions, may be subsequently made to alter the characteristics of the polypeptide.

C. Modification of a Fibrinogen γC Coding Sequence

The amino acid sequence of a fibrinogen γC polypeptide, e.g., SEQ ID NO:3, 4, or 5, may be modified while maintaining or enhancing the polypeptide's capability to inhibit endothelial cell proliferation, as determined by the in vitro or in vivo methods described below. Possible modifications to the amino acid sequence of a fibrinogen γC polypeptide may include conservative substitutions; deletion or addition of one or more amino acid residues (e.g., addition at one terminal of the polypeptide of a tag sequence such as 6× His to facilitate purification or identification); truncation of a fragment ranging from approximately 10, 20, 40, 60, 80, or 100 amino acids of the fibrinogen γC polypeptide at either or both of the N— and C-termini; and truncation of a fragment ranging from approximately 10, 20, 40, 60, 80, or 100 amino acids within the fibrinogen γC polypeptide. The general strategy for making these truncation modifications is, in one series, from the N-terminus starting with the smallest truncation gradually increasing in length up to approximately 100 amino acids, and in another series, from the C-terminus starting with the smallest truncation gradually increasing in length up to approximately 100 amino acids. Upon testing the functionality of the truncated polypeptides so generated in an in vitro or in vivo assay and depending on the results of such testing, one may generate additional fibrinogen γC polypeptides with both N— and C-terminal truncations or with internal truncation(s) and further examine their ability to inhibit endothelial cell proliferation.

Several examples of such modified γC amino acid sequence include those generally corresponding to SEQ ID NO:3 with a deletion of at least 4 and up to 20 amino acids at the C-terminus of SEQ ID NO:3. For instance, the present inventors have discovered that the 1-249 fragment of SEQ ID NO:3 is particularly effective in inducing endothelial cell apoptosis in in vitro assays.

A variety of mutation-generating protocols are established and described in the art, and can be readily used to modify a polynucleotide sequence encoding a fibrinogen γC polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

D. Modification of Nucleic Acids for Preferred Codon Usage in a Host Organism

The polynucleotide sequence encoding a fibrinogen γC polypeptide can be further altered to coincide with the preferred codon usage of a particular host. For example, the preferred codon usage of one strain of bacterial cells can be used to derive a polynucleotide that encodes a fibrinogen γC polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the fibrinogen γC-related polypeptides.

E. Chemical Synthesis of Fibrinogen γC

The amino acid sequence of fibrinogen γ chain, including several isoforms, has been established (e.g., GenBank Accession Nos. P02679, AAH07044, AAK19751, AAB59531, and AAP35744). The crystal structure of the C-terminal region of the γ chain has been described and the amino acid sequence of this region is set forth in, e.g., GenBank Accession Nos. 1FIB, 1FICB, and 1FID, or as shown in SEQ ID NO:3, SEQ ID NO:4, or SEQ ID NO:6.

As discussed above, the amino acid sequence of a fibrinogen γC polypeptide may also be modified without compromising its ability to inhibit endothelial cell proliferation. The fibrinogen γC-related polypeptides of the present invention thus can also be synthesized chemically using conventional peptide synthesis or other protocols well known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

IV. Expression and Purification of Fibrinogen γC-Related Polypeptides

Following verification of the coding sequence, the fibrinogen γC-related polypeptide of the present invention can be produced using routine techniques in the field of recombinant genetics, relying on the polynucleotide sequences encoding the polypeptide disclosed herein.

A. Expression Systems

To obtain high level expression of a nucleic acid encoding a fibrinogen γ-related polypeptide of the present invention, one typically subclones a polynucleotide encoding the fibrinogen γC polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the fibrinogen γC polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the fibrinogen γC-related polypeptide in host cells. A typical expression cassette thus contains a promoter operably linked to the nucleic acid sequence encoding the fibrinogen γC polypeptide and signals required for efficient polyadenylation of the transcript, ribosome binding sites, and translation termination. The nucleic acid sequence encoding the fibrinogen γC polypeptide is typically linked to a cleavable signal peptide sequence to promote secretion of the fibrinogen γC polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Róus sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the fibrinogen γC-related polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a fibrinogen γC-related polypeptide of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to any wild-type or modified fibrinogen γC fragment or its coding sequence while still retaining the biological activity of the fibrinogen γC polypeptide, e.g., the inhibitory effect toward endothelial cell proliferation. Moreover, modifications of a polynucleotide coding sequence may also be made to accommodate preferred codon usage in a particular expression host without altering the resulting amino acid sequence.

B. Transfection Methods

Standard transfection methods are used to produce bacterial, mammalian, yeast, insect, or plant cell lines that express large quantities of a fibrinogen γC polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the fibrinogen γC polypeptide.

C. Detection of Recombinant Expression of a γC-Related Polypeptide in Host Cells

After the expression vector is introduced into appropriate host cells, the transfected cells are cultured under conditions favoring expression of the fibrinogen γC-related polypeptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a fibrinogen γC polypeptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a fibrinogen γC polypeptide of the present invention, such as a polypeptide having the amino acid sequence of SEQ ID NO:3, (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against fibrinogen γC polypeptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976). More detailed descriptions of preparing antibodies against the fibrinogen γC polypeptide of the present invention and conducting immunological assays detecting the fibrinogen γC polypeptide are provided in a later section.

D. Purification of Recombinantly Produced γC-Related Polypeptides

Once the expression of a recombinant fibrinogen γC polypeptide in transfected host cells is confirmed, the host cells are then cultured in an appropriate scale for the purpose of purifying the recombinant polypeptide.

1. Purification of Recombinantly Produced γC Polypeptide from Bacteria

When the fibrinogen γC-related polypeptides of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a fibrinogen γC polypeptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide, e.g., the fibrinogen γC-related polypeptide of the present invention, is expressed in host cells in a soluble form, its purification can follow the standard protein purification procedure described below. This standard purification procedure is also suitable for purifying fibrinogen γC polypeptides obtained from chemical synthesis or an enzymatic digestion of a fibrinogen gamma chain.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a fibrinogen γC-related polypeptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a fibrinogen γC polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a fibrinogen γC polypeptide of the present invention) can also be separated from other proteins on the basis of their size, net surface charge, hydrophobicity, or affinity for ligands. In addition, antibodies raised against a fibrinogen γC fragment can be conjugated to column matrices and the fibrinogen γ polypeptide immunopurified. All of these methods are well known in the art.

It will be apparent to one of skill that chromatographic techniques can be performed at any scale and using equipment from many different manufacturers (e.g., Pharmacia Biotech).

V. Immunoassays for Detection of Recombinant Fibrinogen γC Expression

To confirm the production of a recombinant fibrinogen γC-related polypeptide, immunological assays may be useful to detect in a sample the expression of the polypeptide. Immunological assays are also useful for quantifying the expression level of the recombinant fibrinogen γC polypeptide. Antibodies against a fibrinogen γC polypeptide are necessary for carrying out these immunological assays.

A. Production of Antibodies against a Fibrinogen γC Polypeptide

Methods for producing polyclonal and monoclonal antibodies that react specifically with an immunogen of interest are known to those of skill in the art (see, e.g., Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a fibrinogen γC polypeptide of the present invention) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. The general principles and methods of recombinant polypeptide production discussed above are applicable for antibody production by recombinant methods.

B. Immunoassays for Detecting Recombinant Fibrinogen γC Expression

Once antibodies specific for a fibrinogen γC polypeptide of the present invention are available, the amount of the polypeptide in a sample, e.g., a cell lysate, can be measured by a variety of immunoassay methods providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

1. Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and calorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

2. Immunoassay Formats

Immunoassays for detecting a target protein of interest (e.g., a fibrinogen γC polypeptide) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a fibrinogen γC polypeptide in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against a fibrinogen γC polypeptide.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

III. Functional Assays

A. In vitro Assays

Following exposure to 0.1-20 μg/ml a fibrinogen γC polypeptide in the tissue culture for 0.5-48 hours, endothelial cells are examined for their proliferation/survival status using methods such as direct cell number counting, BrdU or H³-thymidine incorporation, tetrazolium salt 3, [4,5-dimethylthiazol-2-yl]-2,5-diphenyltetrazolium bromide (MTT) cell proliferation assay, 3-(4,5-dimethylthiazol-2-yl)-5-(3-carboxymethoxyphenyl)-2-(4-sulfophenyl)-2H-tetrazolium (MTS) cell proliferation assay, chicken embryo allantoic membrane (CAM) assay, TUNNEL assay, annexin V binding assay, etc. An inhibitory effect is detected when a statistically significant decrease in cell proliferation is found to be at least 10%, more preferably at least 20%,30%,40%, or 50%. Similarly, any statistically significant increase in programmed cell death of at least 10%, 20%, 30%, 40%, or 50% is recognized as a positive effect on promoting cell death.

B. In vivo Assays

The inhibitory effects of a fibrinogen γC polypeptide of the present invention can also be demonstrated in in vivo assays. For example, Chinese hamster ovarian (CHO) cells that have been transfected with an expression cassette comprising a polynucleotide encoding the fibrinogen γC polypeptide and express a secreted form of the polypeptide can be injected into animals with a compromised immune system (e.g., nude mice, SCID mice, or NOD/SCID mice). Tumor development is monitored in comparison with a control group of animals who received only cancer cells transfected with the expression cassette without the fibrinogen coding sequence. An inhibitory effect is detected when a statistically significant negative effect on tumor growth is established in the test group. Preferably, the negative effect is at least a 10% decrease; more preferably, the decrease is at least 20%, 30%, 40%, 50%, 60%, 70%, 80%, or 90%.

IV. Pharmaceutical Compositions and Administration

The present invention also provides pharmaceutical compositions comprising an effective amount of a fibrinogen γC-related polypeptide or a polynucleotide encoding a fibrinogen γC-related polypeptide for inhibiting endothelial cell proliferation in both prophylactic and therapeutic applications. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intraperitoneal. The preferred routes of administering the pharmaceutical compositions are local delivery to an organ or tissue suffering from a condition exacerbated by the proliferation of endothelial cells (e.g., a tumor) at daily doses of about 0.01-5000 mg, preferably 5-500 mg, of a fibrinogen γC polypeptide for a 70 kg adult human per day. The appropriate dose may be administered in a single daily dose or as divided doses presented at appropriate intervals, for example as two, three, four, or more subdoses per day.

For preparing pharmaceutical compositions containing a fibrinogen γC polypeptide, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a fibrinogen γC-related polypeptide. In tablets, the active ingredient (fibrinogen γC-related polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient of fibrinogen γC-related polypeptide. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a fibrinogen γC-related polypeptide with encapsulating material as a carrier providing a capsule in which the fibrinogen γC-related polypeptide (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a fibrinogen γC-related polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a fibrinogen γC-related polypeptide) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing fibrinogen γC-related polypeptides can be administered for prophylactic and/or therapeutic treatments. In therapeutic applications, compositions are administered to a patient already suffering from a condition that may be exacerbated by the proliferation of endothelial cells, e.g., angiogenesis supporting tumor growth, in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the fibrinogen γC-related polypeptide per day for a 70 kg patient, with dosages of from about 5 mg to about 500 mg of the polypeptide per day for a 70 kg patient being more commonly used.

In prophylactic applications, pharmaceutical compositions containing fibrinogen γC-related polypeptides are administered to a patient susceptible to or otherwise at risk of developing a disease or condition in which endothelial cell proliferation is undesirable, in an amount sufficient to delay or prevent the onset of the symptoms. Such an amount is defined to be a “prophylactically effective dose.” In this use, the precise amounts of the fibrinogen γC-related polypeptide again depend on the patient's state of health and weight, but generally range from about 0.1 mg to about 2,000 mg of the polypeptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a fibrinogen γC-related polypeptide sufficient to effectively inhibit endothelial cell proliferation in the patient, either therapeutically or prophylatically.

V. Therapeutic Applications Using Nucleic Acids

A variety of diseases can be treated by therapeutic approaches that involve introducing a nucleic acid encoding a fibrinogen γC-related polypeptide of the present invention into a cell such that the coding sequence is transcribed and the fibrinogen γC-related polypeptide is produced in the cell. Diseases amenable to treatment by this approach include a broad spectrum of solid tumors, the survival and growth of which rely on the continued blood supply and thus require the formation of new blood vessels. For discussions on the application of gene therapy towards the treatment of genetic as well as acquired diseases, see, Miller Nature 357:455-460 (1992); and Mulligan Science 260:926-932 (1993).

A. Vectors for Gene Delivery

For delivery to a cell or organism, a polynucleotide encoding a fibrinogen γC polypeptide of the invention can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In a preferred embodiment, the polynucleotide encoding a fibrinogen γC-related polypeptide can be operably linked to expression and control sequences that can direct expression of the polypeptide in the desired target host cells. Thus, one can achieve expression of the fibrinogen γC-related polypeptide under appropriate conditions in the target cell.

B. Gene Delivery Systems

Viral vector systems useful in the expression of a fibrinogen γC-related polypeptide include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest (e.g., one encoding for a fibrinogen γC-related polypeptide of the present invention) are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a fibrinogen γC-related polypeptide of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

C. Pharmaceutical Formulations

When used for pharmaceutical purposes, the nucleic acid encoding a fibrinogen γC polypeptide is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

D. Administration of Formulations

The formulations containing a nucleic acid encoding a fibrinogen γC-related polypeptide of the invention can be delivered to any tissue or organ using any delivery method known to the ordinarily skilled artisan. In some embodiments of the invention, the nucleic acids encoding fibrinogen γC-related polypeptides are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the nucleic acid of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, ultrasound, electroporation, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg DNA per patient are typical. Doses generally range between about 0.01 and about 50 mg per kilogram of body weight, preferably between about 0.1 and about 5 mg/kg of body weight or about 10⁸-10¹⁰ or 10¹² particles per injection. In general, the dose equivalent of a naked nucleic acid from a vector is from about 1 μg-100 μg for a typical 70 kg patient, and doses of vectors which include a retroviral particle are calculated to yield an equivalent amount of nucleic acid encoding a fibrinogen γC-related polypeptide.

VI. Kits

The invention also provides kits for inhibiting endothelial cell proliferation according to the method of the present invention. The kits typically include a container that contains a pharmaceutical composition having an effective amount of a fibrinogen γC-related polypeptide or a polynucleotide sequence encoding a fibrinogen γC-related polypeptide, as well as informational material containing instructions on how to dispense the pharmaceutical composition, including description of the type of patients who may be treated (e.g., a person at risk of developing advanced tumor mass), the schedule (e.g., dose and frequency) and route of administration, and the like.

### EXAMPLES

The following examples are provided by way of illustration only and not by way of limitation. Those of skill in the art will readily recognize a variety of non-critical parameters that could be changed or modified to yield essentially similar results.

### Example 1

### Fibrinogen γC Induces Apoptosis of Endothelial Cells

Fibrinogen γ-chain C-terminal domain (designated γC, 30 Kd, about 250 amino acid residues, sequence as set forth in SEQ ID NO:3) contains the major binding sites for integrin αvβ3. In contrast to native fibrinogen, which generates proliferative signals upon binding to integrins, γC effectively blocked proliferation of cultured bovine artery endothelial (BAE) cells. γC induced apoptosis of BAE cells was demonstrated in the annexin V binding assay.

Since γC induced massive MAP kinase activation, it is likely that γC actively transduces intracellular signals that lead to apoptosis (rather than blocking binding of cells to other integrin ligands). This observation is novel and important for understanding signals from fibrinogen, and particularly useful for developing new anti-angiogenic strategies.

γC Inhibits Endothelial Cell Proliferation.

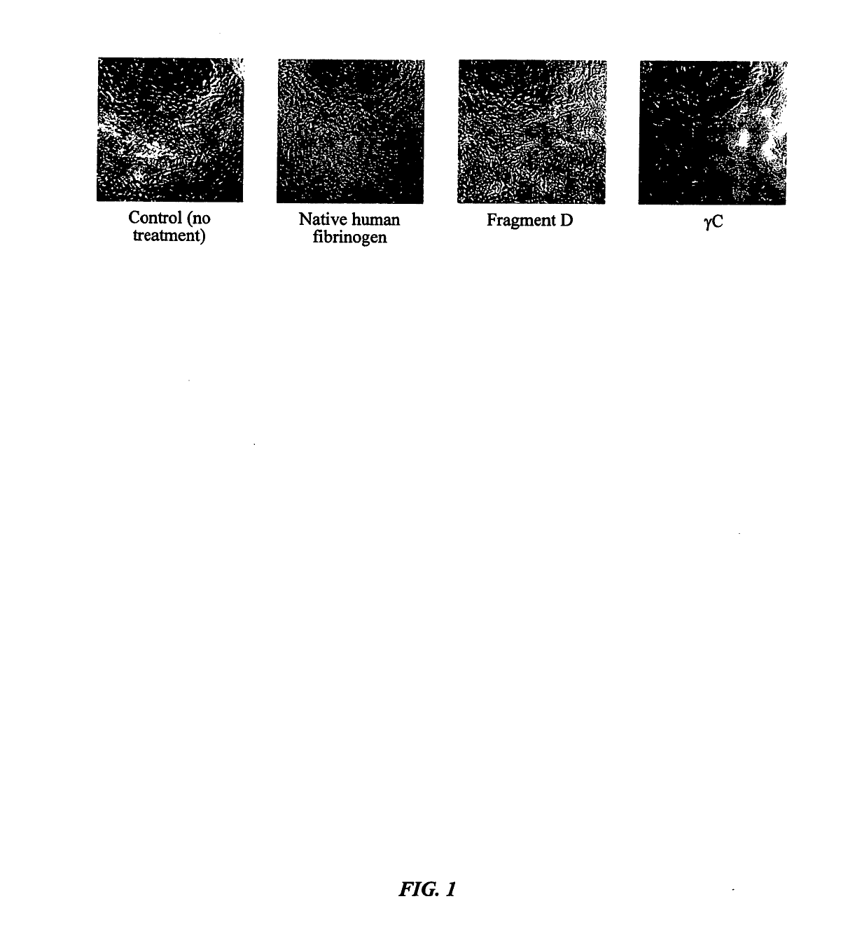

Isolated γC domain (which is part of fragment D) blocked proliferation of BAE cells at concentrations of less than 1 μg/ml (FIG. 1). Native fibrinogen or fragment D did not affect proliferation of BAE cells under the conditions used.

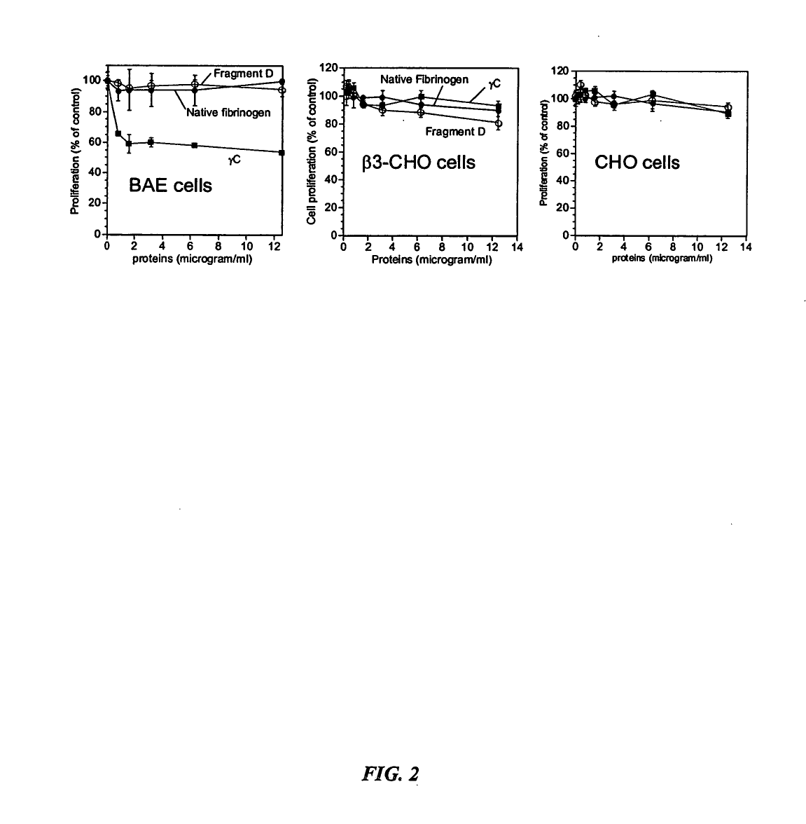

The γC domain also blocked proliferation of BAE cells in the MTS assay (FIG. 2). This suppression of proliferation was observed in BAE cells, but not in CHO or β3-CHO cells, indicating that γC's anti-proliferative effect is specific to endothelial cells.

γC Induces BAE Cells Apoptosis

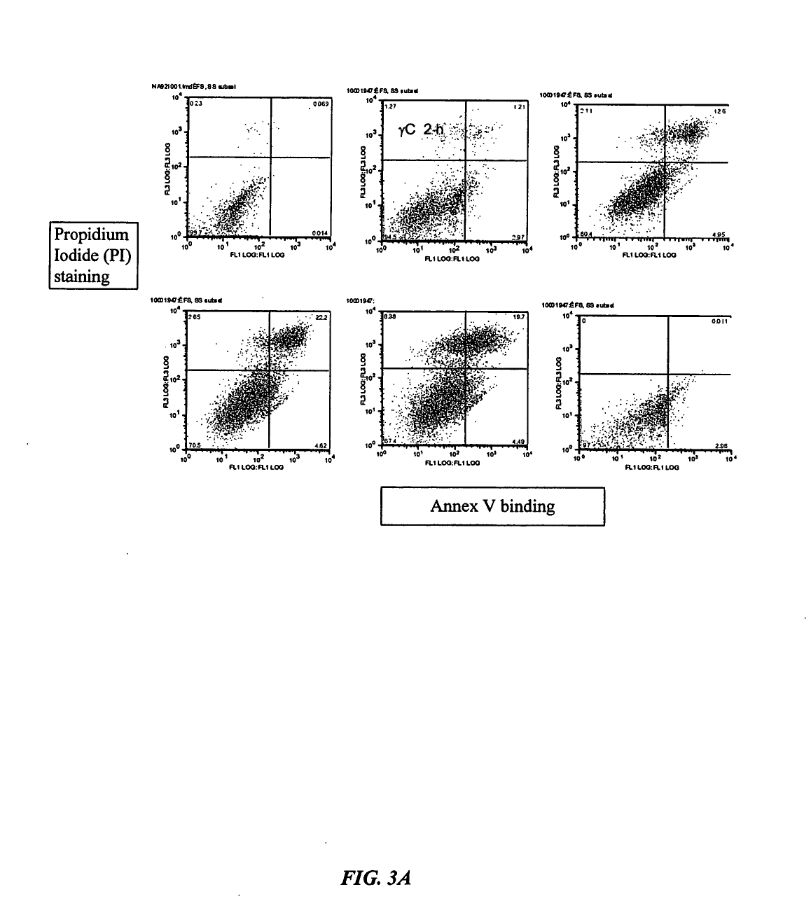

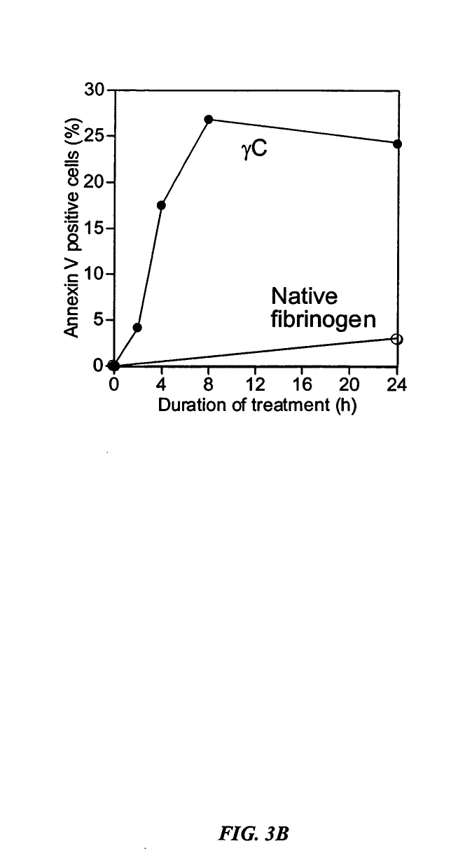

γC domain induced apoptosis of BAE cells in 2-4 hours (FIG. 3) as detected by annexin V binding assays. In contrast, native fibrinogen did not show the same effects.

γC Induces MAP Kinase Activation

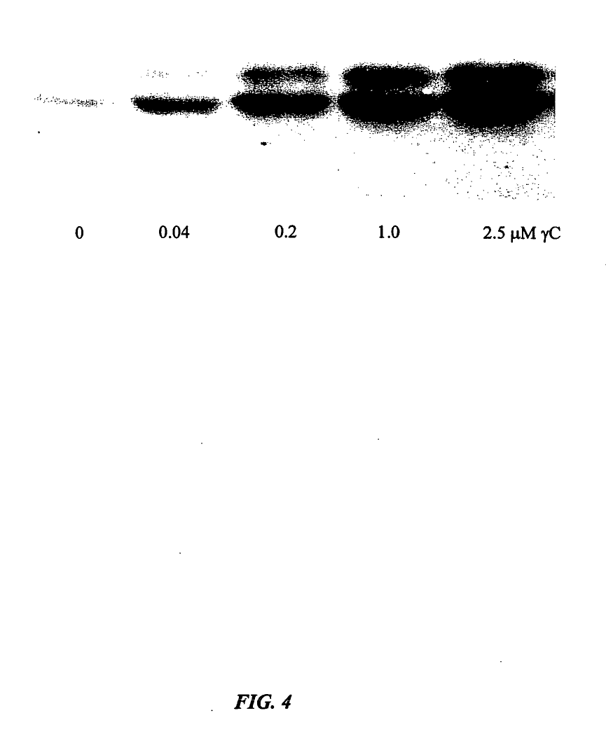

It was shown that soluble γC strongly induced MAP kinase activation at very low concentrations (less than 1 μg/ml in the medium). This suggests that γC-induced apoptosis may be due to direct effect of γC-induced intracellular signals rather than due to blocking of cell-extracellular interaction (FIG. 4). It remains a possibility that γC acts as an antagonist and blocks signaling from other integrin ligands by competing for binding to integrins.

γC Induces Apoptosis in CPAE Cells and Activation of Caspases

Fibrinogen γC induced apoptosis in calf pulmonary artery endothelial (CPAE) cells. The activation of caspase-3 and caspase-7 by γC, both at the level of increased protein expression and at the level of increased enzymatic activity, suggests that γC-induced endothelial cell apoptosis is mediated by caspases.

### Example 2

### Fibrinogen γC Suppresses Tumor Growth in Animals

The human xenograft mouse as described by Yonou et al. (Cancer Res., 2001, 61(5):2177-2182) is used as an animal model to demonstrate fibrinogen γC's tumor suppression activity in vivo. Human breast adenocarcinoma BT20 cells are subcutaneously injected into 7-9-week-old male non-obese diabetes/severe combined immunodeficiency (NOD/SCID) mice. The animals are divided into 4 groups, each having 5 animals. Each animal receives 2×10⁶ cells in the injection. The treatment groups, Groups 1-3, are injected daily intraperitoneally with recombinant γC. The control group, Group 4, receives injections of equal volumes of PBS each day. The dimensions of the tumors are measured and the tumor volumes are calculated. After two weeks of treatment, the animals are sacrificed and the tumors are excised, sectioned, and evaluated histologically. To examine the tumor vasculatures, the tumor sections are stained by CD31 anti-mouse monoclonal antibody (PharMingen, San Diego, Calif.). Tumor size and the extent of tumor vasculature are compared among all treatment and control groups.

### Example 3

### Tumorigenicity of Cancer Cells Expressing Secreted γC

Chinese hamster ovarian (CHO) cells are first transfected with secretion vector pSECtag that directs the expression and secretion of fibrinogen γC. Control cells are also established by transfecting CHO cells with the empty pSECtag vector without the coding sequence for γC. Upon establishing the transfected cancer cells, the cells are introduced into the NOD/SID mice as described in Example 2. The tumor volume is monitored two to three times weekly by measuring the three dimensions of the tumors in the animals of both the experiment and control groups. Two weeks after the beginning of the experiments, all animals are sacrificed and tumors are excised and examined for their volume and vasculature as described in Example 2.

### Example 4

### Inhibition of Endothelial Cell Proliferation by γC-399tr

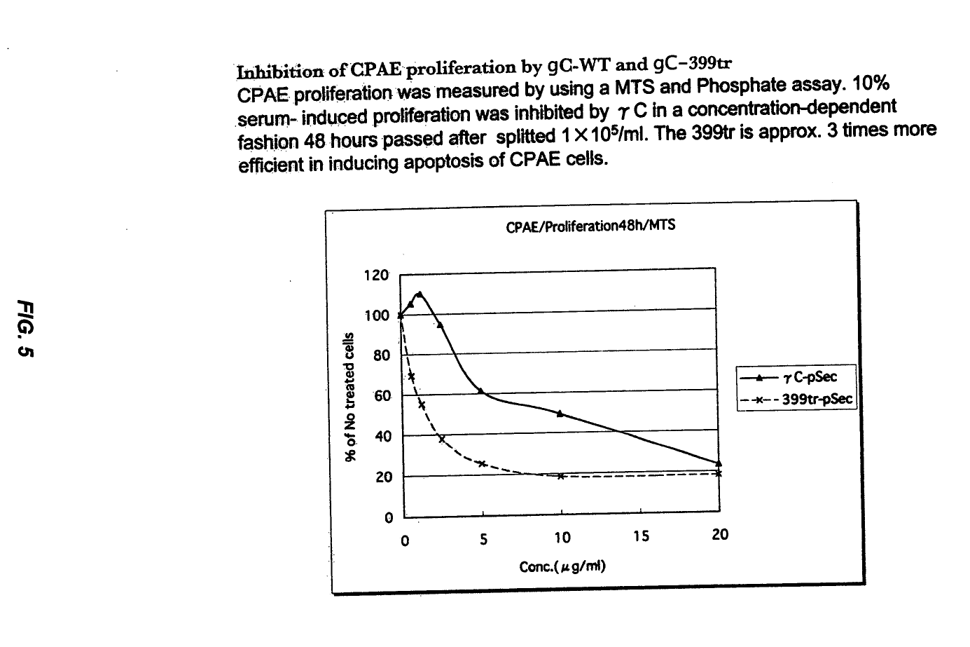

A deletion mutant of γC that has 12 amino acids truncated from the C-terminal of γC, termed γC-399tr (having the amino acid sequence of 1-249 of SEQ ID NO:3), was recombinantly produced and purified. The effects of this mutant on CPAE proliferation were tested along with a wild-type γC (FIG. 5). γC-399tr was found to be surprisingly effective to induce apoptosis in CPAE cells, about 3 times the efficacy of wild-type γC.

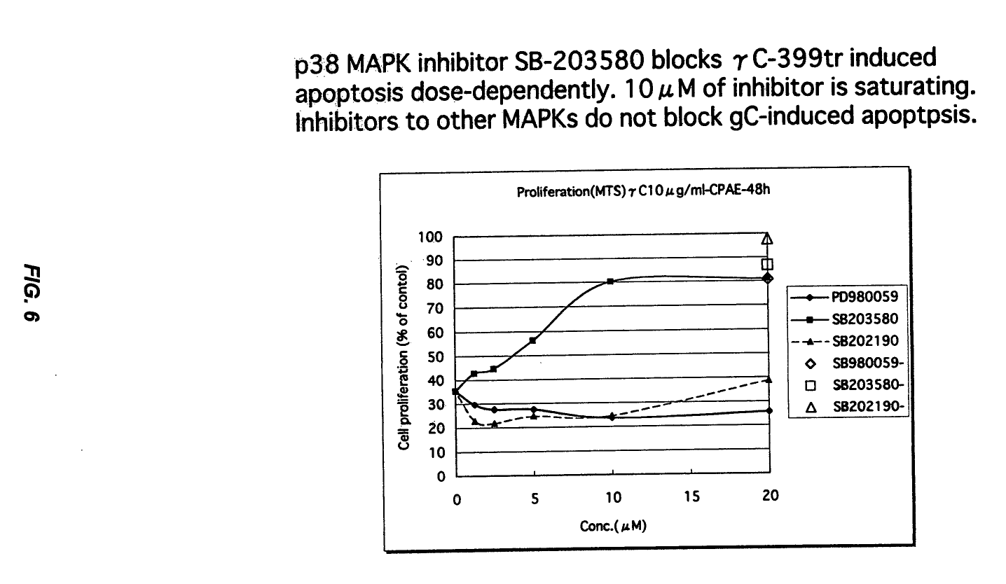

It was further observed that endothelial cell apoptosis induced by γC-399tr can be blocked by p38 MAP kinase inhibitor SB-203580 but not inhibitors of some other MAP kinases. FIG. 6 shows some examples of MAPK inhibitors and their ability in blocking γC-399tr-induced apoptosis.

### Example 5

### γC-399tr Suppresses Tumor Growth in Animals

Following the general procedure described in Example 2, DLD-1 human colon adenocarcinoma cells were injected into SCID mice. Subsequently, the treatment groups of mice received injections of recombinant γC-399tr in PBS, whereas the control group received PBS injections. Throughout the experiments, tumor size was compared between the treatment groups and the control group. γC-399tr treatment consistently led to reduced tumor size.

All patents, patent applications, and other publications, including GenBank Accession Numbers, cited in this application are incorporated by reference in the entirety for all purposes.

What is claimed is:

1. A composition comprising a polypeptide and a pharmaceutically acceptable carrier, wherein the polypeptide: (a) comprises an amino acid sequence having at least 90% sequence identity to the full length of SEQ ID NO:3, 4, or 6; and (b) inhibits endothelial cell proliferation.

2. The composition of claim 1, wherein the polypeptide inhibits endothelial cell proliferation in an in vitro assay.

3. The composition of claim 1, wherein the amino acid sequence is SEQ ID NO:3.

4. The composition of claim 1, wherein the amino acid sequence is SEQ ID NO:4.

5. The composition of claim 1, wherein the amino acid sequence is SEQ ID NO:6.

6. The composition of claim 1, wherein the amino acid sequence is the amino acid residues 1-249 of SEQ ID NO:3.

7. A composition comprising a nucleic acid and a pharmaceutically acceptable carrier, wherein the nucleic acid comprises a polynucleotide sequence, which encodes a polypeptide that: (a) comprises an amino acid sequence having at least 90% sequence identity to the full length of SEQ ID NO:3, 4, or 6; and (b) inhibits endothelial cell proliferation.

8. The composition of claim 7, wherein the polypeptide inhibits endothelial cell proliferation in an in vitro assay.

9. The composition of claim 7, wherein the amino acid sequence is SEQ ID NO:3.

10. The composition of claim 7, wherein the amino acid sequence is SEQ ID NO:4.

11. The composition of claim 7, wherein the amino acid sequence is SEQ ID NO:6.

12. The composition of claim 7, wherein the amino acid sequence is the amino acid residues 1-249 of SEQ ID NO:3.

13. The composition of claim 7, wherein the nucleic acid is an expression cassette.

14. A method for inhibiting endothelial cell proliferation, comprising the step of contacting an endothelial cell an effective amount of a polypeptide, wherein the polypeptide: (a) comprises an amino acid sequence having at least 90% sequence identity to the full length of SEQ ID NO:3, 4, or 6; and (b) inhibits endothelial cell proliferation.

15. A method for inhibiting endothelial cell proliferation in a subject, comprising the step of administering to the subject an effective amount of a polypeptide, wherein the polypeptide: (a) comprises an amino acid sequence having at least 90% sequence identity to the full length of SEQ ID NO:3, 4, or 6; and (b) inhibits endothelial cell proliferation.

16. The method of claim 14 or 15, wherein the polypeptide inhibits endothelial cell proliferation in an in vitro assay.

17. The method of claim 14 or 15, wherein the amino acid sequence is SEQ ID NO:3.

18. The method of claim 14 or 15, wherein the amino acid sequence is SEQ ID NO:4.

19. The method of claim 14 or 15, wherein the amino acid sequence is SEQ ID NO:6.

20. The method of claim 14 or 15, wherein the amino acid sequence is the amino acid residues 1-249 of SEQ ID NO:3.

21. The method of claim 15, wherein the administering is performed locally.

22. A kit for inhibiting endothelial cell proliferation, comprising the composition of claim 1.

23. A method for inhibiting endothelial cell proliferation, comprising the step of contacting an endothelial cell an effective amount of a nucleic acid comprising a polynucleotide, which encodes a polypeptide that: (a) comprises an amino acid sequence having at least 90% sequence identity to the full length of SEQ ID NO:3, 4, or 6; and (b) inhibits endothelial cell proliferation.

24. A method for inhibiting endothelial cell proliferation in a patient, comprising the step of administering to the patient an effective amount of a nucleic acid comprising a polynucleotide, which encodes a polypeptide that: (a) comprises an amino acid sequence having at least 90% sequence identity to the full length of SEQ ID NO:3, 4, or 6; and (b) inhibits endothelial cell proliferation.

25. The method of claim 23 or 24, wherein the polypeptide inhibits endothelial cell proliferation in an in vitro assay.

26. The method of claim 23 or 24, wherein the amino acid sequence is SEQ ID NO:3.

27. The method of claim 23 or 24, wherein the amino acid sequence is SEQ ID NO:4.

28. The method of claim 23 or 24, wherein the amino acid sequence is SEQ ID NO:6.

29. The method of claim 23 or 24, wherein the amino acid sequence is the amino acid residues 1-249 of SEQ ID NO:3.

30. The method of claim 24, wherein the administering is performed locally.

31. A kit for inhibiting endothelial cell proliferation, comprising the composition of claim 7.


SEQUENCE LISTING (7 sequences)

SEQ ID NO: 1 - DNA, 1594 bp, Homo sapiens
  fibrinogen gamma chain, transcript splicing variant gamma-A cDNA
acagtgctgacactacaaggctcggagctccgggcactcagacatcatgagttggtccttgcacccccggaatttaattctctacttctatgctcttttatttctctcttcaacatgtgtagcatatgttgctaccagagacaactgctgcatcttagatgaaagattcggtagttattgtccaactacctgtggcattgcagatttcctgtctacttatcaaaccaaagtagacaaggatctacagtctttggaagacatcttacatcaagttgaaaacaaaacatcagaagtcaaacagctgataaaagcaatccaactcacttataatcctgatgaatcatcaaaaccaaatatgatagacgctgctactttgaagtccaggaaaatgttagaagaaattatgaaatatgaagcatcgattttaacacatgactcaagtattcgatatttgcaggaaatatataattcaaataatcaaaagattgttaacctgaaagagaaggtagcccagcttgaagcacagtgccaggaaccttgcaaagacacggtgcaaatccatgatatcactgggaaagattgtcaagacattgccaataagggagctaaacagagcgggctttactttattaaacctctgaaagctaaccagcaattcttagtctactgtgaaatcgatgggtctggaaatggatggactgtgtttcagaagagacttgatggcagtgtagatttcaagaaaaactggattcaatataaagaaggatttggacatctgtctcctactggcacaacagaattttggctgggaaatgagaagattcatttgataagcacacagtctgccatcccatatgcattaagagtggaactggaagactggaatggcagaaccagtactgcagactatgccatgttcaaggtgggacctgaagctgacaagtaccgcctaacatatgcctacttcgctggtggggatgctggagatgcctttgatggctttgattttggcgatgatcctagtgacaagtttttcacatcccataatggcatgcagttcagtacctgggacaatgacaatgataagtttgaaggcaactgtgctgaacaggatggatctggttggtggatgaacaagtgtcacgctggccatctcaatggagtttattaccaaggtggcacttactcaaaagcatctactcctaatggttatgataatggcattatttgggccacttggaaaacccggtggtattccatgaagaaaaccactatgaagataatcccattcaacagactcacaattggagaaggacagcaacaccacctggggggagccaaacaggctggagacgtttaaaagaccgtttcaaaagagatttacttttttaaaggactttatctgaacagagagatataatatttttcctattggacaatggacttgcaaagcttcacttcattttaagagcaaaagaccccatgttgaaaactccataacagttttatgctgatgataatttatctacatgcatttcaataaaccttttgtttcctaagacgaaaaaaaaaaaaaaaaaaaaaaaaaaaaaaa

SEQ ID NO: 2 - protein, 437 aa, Homo sapiens
  fibrinogen gamma chain, isoform gamma-A precursor
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 3 - protein, 261 aa, Homo sapiens
  C-terminal region of fibrinogen gamma chain, isoform gamma-A, residues 177-437
KDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 4 - protein, 269 aa, Homo sapiens
  C-terminal region of fibrinogen gamma chain, isoform gamma-A, residues 169-437
VQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 5 - protein, 453 aa, Homo sapiens
  fibrinogen gamma chain, isoform gamma-B precursor
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 6 - protein, 277 aa, Homo sapiens
  C-terminal region of fibrinogen gamma chain, isoform gamma-B, residues 177-453
KDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 7 - protein, 4 aa, Artificial Sequence
  Description of Artificial Sequence4 amino acids deleted from C-terminus of fibrinogen gamma chain, isoform gamma-A core gammaC sequence
AGDV